(19) United States
(12) Patent Application Publication
(10) Pub. No.: US 2010/0030164 A1
(43) Pub. Date: Feb. 4, 2010

(54) Medical Valve with Raised Seal

(75) Inventors: Ian Kimball, Clinton, MA (US); Todd S. Vangsness, Stow, MA (US)

Correspondence Address: Sunstein Kann Murphy & Timbers LLP, 125 SUMMER STREET, BOSTON, MA, 02110-1618, US

(73) Assignee: NP MEDICAL INC., Clinton, MA (US)

(21) Appl. No.: 12/535,059

(22) Filed: Aug. 4, 2009

Related U.S. Application Data
(60) Provisional application No. 61/169,903, filed on Apr. 16, 2009.
(60) Provisional application No. 61/085,984, filed on Aug. 4, 2008.

(51) Int. Cl. A61M 39/06

(52) U.S. Cl. 604/256

(57) ABSTRACT

A seal for a medical valve having a housing and an inlet includes a seal portion and a sleeve portion. The seal portion may be over the inlet of the valve and have a normally closed aperture therethrough. The aperture may be located proximal to the inlet. The sleeve portion may extend distally from the seal portion, and at least a part of the sleeve portion may be chemically bonded to the inlet.

### PRIORITY

This patent application claims priority from provisional United States patent applications:

Application No. 61/085,984 filed Aug. 4, 2008, entitled, “Raised Inlet Seal for Medical Valve,” assigned attorney docket number 1600/199, and naming Ian Kimball and Todd S. Vangsness as inventors, the disclosure of which is incorporated herein, in its entirety, by reference.

Application No. 61/169,903, filed Apr. 16, 2009, entitled, “Raised Inlet Seal for Medical Valve,” assigned attorney docket number 1600/A05, and naming Ian Kimball and Todd S. Vangsness as inventors, the disclosure of which is incorporated herein, in its entirety, by reference.

### TECHNICAL FIELD

The present invention relates to medical valves. More specifically, the invention relates to inlet seals for medical valves.

### BACKGROUND ART

In general terms, medical valving devices often act as a sealed port that may be repeatedly accessed to non-invasively inject fluid into (or withdraw fluid from) a patient's vasculature. During use, medical personnel may insert a luer tip syringe into the proximal port of a properly secured medical valve to inject fluid into (or withdraw fluid from) a patient. Once inserted, the syringe may freely inject or withdraw fluid to and from the patient.

### SUMMARY OF THE INVENTION

In accordance with one embodiment of the present invention, a seal for a medical valve having a housing and an inlet includes a seal portion over the inlet of the valve and a sleeve portion. The seal portion may have a normally closed aperture (e.g., a slit or a pinhole) through the seal portion and located proximal to the inlet. The sleeve portion may extend distally from the seal portion, and may extend into the inner diameter of the inlet. At least a part of the sleeve portion and/or the seal portion may be chemically bonded to the inlet, for example, using an over-mold, two-shot, or co-injection manufacturing process, collectively known in the art as multimaterial molding. The seal portion and/or the sleeve portion may be resilient and deform into the inlet after insertion of a medical instrument

The seal may also include at least one cross member extending across a diameter of the seal portion. The cross-member(s) may be located on a distal surface of the seal portion, and may aid the seal and aperture in preventing microbial ingress into the valve. Additionally or alternatively, the seal may also include a lip portion that is co-centric with the sleeve portion and extend distally along an outer diameter of the inlet. The seal portion may be swabbable.

In accordance with other embodiments of the present invention, a seal for a medical valve having a housing and an inlet may include a seal portion and a normally closed aperture (e.g., a slit). The seal portion may be chemically bonded to the housing and may be disposed over and raised above the inlet of the valve. The normally closed aperture may extend through the seal portion and may be located proximal to the inlet.

The seal may also include a sleeve portion. The sleeve portion may extend distal to the seal portion and may be disposed within an inner diameter of the inlet. At least a portion of the sleeve portion and/or the seal portion may be chemically bonded to the inlet. The seal portion and the sleeve portion may be chemically bonded to the inlet using a multimaterial molding process. The seal may be resilient.

In accordance with still further embodiments of the present invention, a medical valve may have an open mode that permits fluid flow, and a closed mode that prevents fluid flow. The medical valve may include an inlet housing with an inlet, and a resilient seal member chemically bonded to the inlet housing. The seal member may have a normally closed aperture. At least a portion of the seal member may extend proximal to the inlet such that the normally closed aperture is proximal to the inlet. The seal member may be chemically bonded to the inlet using a multimaterial molding process. The seal member may also include a seal portion disposed over the inlet of the valve, and a sleeve portion extending distally from the seal portion. The sleeve portion may have an outer diameter that is chemically bonded to an inner diameter of the inlet.

In accordance with additional embodiments, a method for producing a medical valve may include inserting a mold into a molding apparatus, injecting a first material into the mold to form an inlet housing of the medical valve, injecting a second material into the mold to form a seal member of the medical valve, and allowing the first and second materials to cure and cool within the mold. The method may also include ejecting the medical valve from the mold, and forming an aperture through the seal member. The aperture may be normally closed and located proximal to the inlet. A portion of the seal member may be chemically bonded to the inlet housing.

The mold may have a first portion and a second portion. The first material may be injected into the first portion, and the second material may be injected into the second portion of the mold. The first and second materials may be allowed to cure at different times. Alternatively, in some embodiments, the first and second material may be injected simultaneously into the same portion of the mold.

### BRIEF DESCRIPTION OF THE DRAWINGS

The foregoing features of the invention will be more readily understood by reference to the following detailed description, taken with reference to the accompanying drawings, in which:

FIG. 1 schematically shows one use of a medical valve configured in accordance with one embodiment of the present invention.

FIG. 2 schematically shows a perspective view of a medical valve configured in accordance with illustrative embodiments of the present invention.

FIG. 3 schematically shows a perspective view of a medical valve inlet housing with inlet seal in accordance with embodiments of the present invention.

FIG. 4 schematically shows a cross-sectional view of the inlet housing shown in FIG. 3 with the inlet seal closed along line 3-3.

FIG. 5 schematically shows a cross-sectional view of the inlet housing shown in FIG. 3 with the inlet seal open and an inserted medical instrument along line 3-3.

FIG. 6 schematically shows an isometric view of the inlet seal in accordance with embodiments of the present invention.

FIG. 7 schematically shows a top view of the inlet seal shown in FIG. 6.

FIG. 8 schematically shows a cross-sectional view of the inlet seal shown in FIG. 6 along line 6-6.

FIG. 9 schematically shows an isometric cross-sectional view of the inlet seal shown in FIG. 6 along line 6-6.

FIG. 10 schematically shows an isometric, bottom angle, cross-sectional view of the inlet seal shown in FIG. 6 along line 6-6.

FIG. 11 schematically shows a bottom view of the inlet seal shown in FIG. 6.

FIG. 12 shows a process of manufacturing the inlet housing and inlet seal shown in FIG. 3 in accordance with various embodiments of the present invention

FIG. 13 schematically shows an alternative embodiment of an inlet seal having a single slit aperture in accordance with embodiments of the present invention.

FIG. 14 schematically shows a cross-sectional view of another alternative embodiment of an inlet seal having alternative internal geometry in accordance with embodiments of the present invention.

FIG. 15 schematically shows a bottom view of the alternative inlet seal of FIG. 14.

FIG. 16A schematically shows a cross-sectional view of a third alternative embodiment of an inlet seal having alternative internal geometry in accordance with embodiments of the present invention.

FIG. 16B schematically shows a cross-sectional perspective view of the third alternative embodiment shown in FIG. 16A, in accordance with embodiments of the present invention.

FIG. 17 schematically shows a cross-sectional view of a fourth alternative embodiment of an inlet seal having alternative internal geometry in accordance with embodiments of the present invention.

FIG. 18 schematically shows a cross-sectional view of a fifth alternative embodiment of an inlet seal having internal geometry with side wall ribs in accordance with embodiments of the present invention.

FIG. 19 schematically shows a bottom view of the fifth alternative embodiment of FIG. 18.

### DETAILED DESCRIPTION OF SPECIFIC EMBODIMENTS

In illustrative embodiments, a medical valve has an inlet seal positioned over and raised above an inlet of the medical valve. Portions of the inlet seal may be chemically bonded to the inlet. Details of illustrative embodiments are discussed in greater detail below.

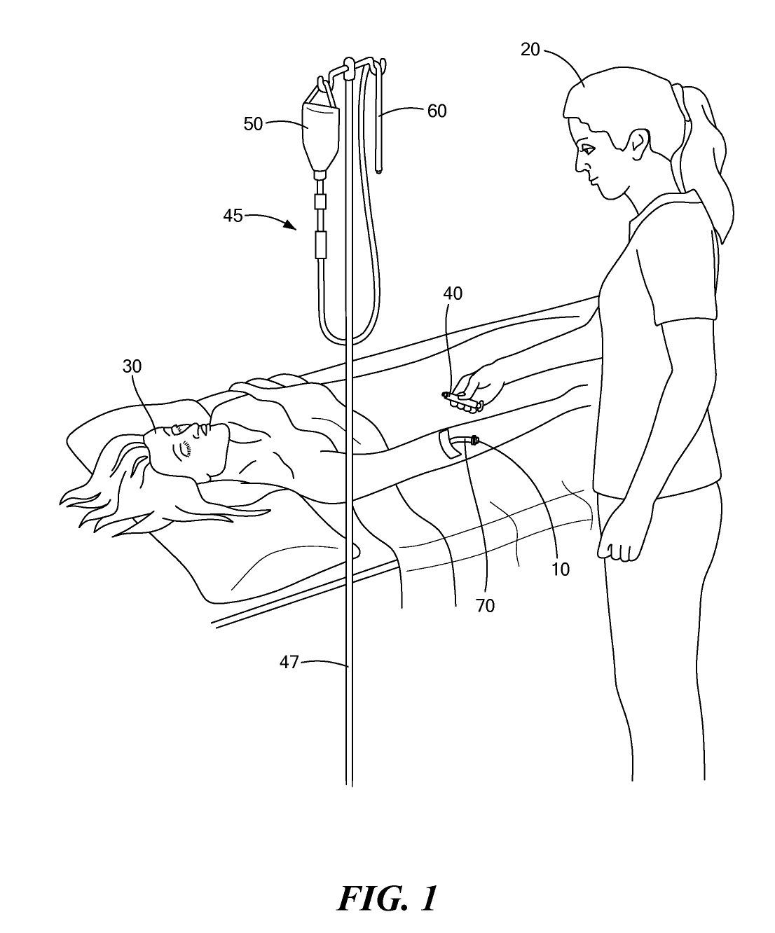

FIG. 1 schematically shows one illustrative use of a medical valve 10 configured in accordance with illustrative embodiments of the invention. In this example, a catheter 70 connects the valve 10 with a patient's vein (the patient is identified by reference number 30). Adhesive tape or similar material may be coupled with the catheter 70 and patient's arm to ensure that the valve remains in place.

After the valve 10 is in place, a nurse, doctor, technician, practitioner, or other user (schematically identified by reference number 20) may intravenously deliver medication to the patient 30, who is lying in a hospital bed. To that end, after the valve is properly primed and flushed (e.g., with a saline flush), the nurse 20 swabs the top surface of the valve 10 to remove contaminants. Next, the nurse 20 uses a medical instrument (e.g., a syringe having a distally located blunt, luer tip complying with ANSI/ISO standards) to inject medication into the patient 30 through the valve 10. For example, the medical practitioner 20 may use the valve 10 to inject drugs such as heparin, antibiotic, pain medication, other intravenous medication, or other fluid deemed medically appropriate. Alternatively, the nurse 20 (or other user) may withdraw blood from the patient 30 through the valve 10.

The medical valve 10 may receive medication or other fluids from other means, such as through a gravity feed system 45. In general, traditional gravity feeding systems 45 often have a bag 50 (or bottle) containing a fluid (e.g., anesthesia medication) to be introduced into the patient 30 hanging from a pole 47. The medical practitioner 20 then connects the bag/bottle 50 to the medical valve 10 using tubing 60 having an attached blunt tip. In illustrative embodiments, the blunt tip of the tubing has a luer taper that complies with the ANSI/ISO standard. After the tubing 60 is connected to the medical valve 10, gravity (or a pump) causes the fluid to begin flowing into the patient 30. In some embodiments, the feeding system 45 may include additional shut-off valves on the tubing 60 (e.g., stop-cock valves or clamps) to stop fluid flow without having to disconnect the tubing 60 from the valve 10. Accordingly, the valve 10 can be used in long-term “indwell” procedures.

After administering or withdrawing fluid from the patient 30, the nurse 20 should appropriately swab and flush the valve 10 and catheter 70 to remove contaminants and ensure proper operation. As known by those skilled in the art, there is a generally accepted valve swabbing and flushing protocol that should mitigate the likelihood of infection. Among other things, as summarized above, this protocol requires proper flushing and swabbing before and after the valve is used to deliver fluid to, or withdraw fluid from the patient.

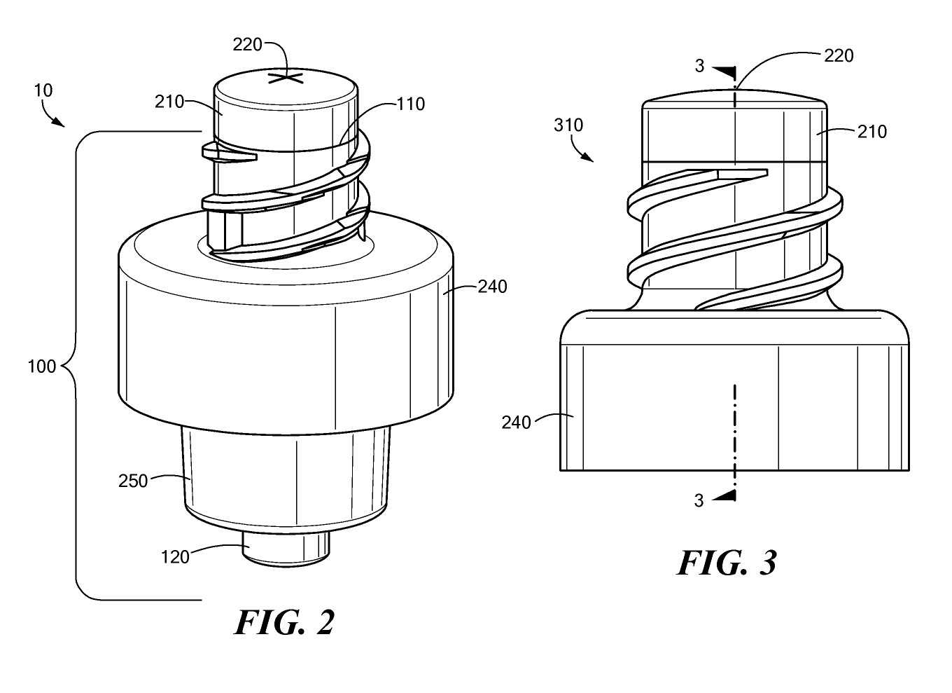

FIG. 2 schematically shows a perspective view of the medical valve 10 shown in FIG. 1. In illustrative embodiments, the medical valve 10 has a housing 100 forming an interior having a proximal port 110 for receiving the instrument 40 and a distal port 120 for injection or withdrawing fluids from the patient. The valve 10 has an open mode that permits fluid flow through the valve 10, and a closed mode that prevents fluid flow through the valve 10. To that end, the interior of the medical valve 10 may contain a valve mechanism (not shown) that selectively controls fluid flow through valve 10.

The valve 10 may also have resilient proximal gland 210 (e.g., an inlet seal). The resilient proximal gland 210 has a resealable aperture 220 that extends entirely through the proximal gland 210. The aperture 220 may, for example, be a pierced hole, or one or more slits (e.g., arranged into a cross, as shown in FIG. 2). Alternatively, the proximal gland 210 may be molded with the aperture 220. As discussed in greater detail below, as the medical instrument 40 is inserted into the valve 10, the proximal gland 210 begins to deform and the aperture 220 opens, allowing the medical instrument 40 to enter the interior of the medical valve through the proximal port 110. If the aperture within the proximal gland 210 is a pinhole (e.g., rather than one or more slits), the medical instrument 40 does not need to penetrate the proximal gland 210. Rather, the medical instrument 40 may deform the proximal gland 210 enough to open the pinhole aperture, but not actually pass through the aperture 220.

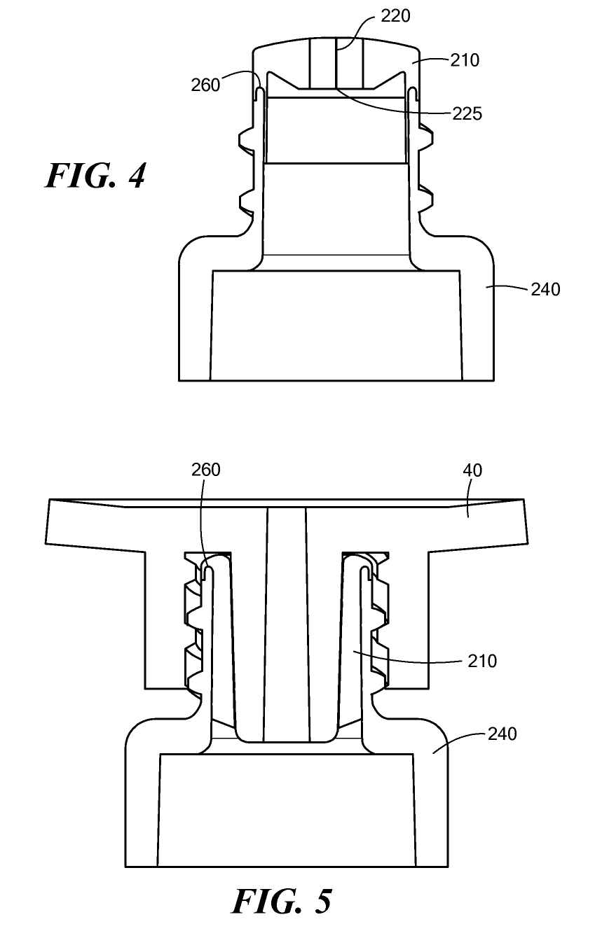

As shown in FIGS. 3-4, the proximal gland 210 may be raised above the exterior inlet face 260 of the inlet housing 240. In such embodiments, the aperture 220 may also be located such that the distal end 225 of the aperture 220 is also raised above the exterior inlet face 260 of the inlet housing 240. As discussed in greater detail below, the raised proximal gland 210 provides a number of benefits, particularly when a multimaterial molding process is used to manufacture the medical valve 10.

As mentioned above and as illustrated in FIG. 5, a medical practitioner may open the medical valve 10 by inserting a medical instrument 40 into the valve 10. In particular, when the medical instrument 40 makes contact with the raised inlet seal 210 and the medical practitioner 20 begins to move the instrument 40 distally, the inlet seal 210 will begin to deform. As the medical instrument 40 is inserted further, the inlet seal 210 will deform into the internal area of the medical valve 10 (e.g., it will deform into the area within the inlet housing 240). As the inlet seal 210 deforms, the aperture 220 opens creating fluid communication between the medical instrument 40 and the internal area of the housing. If the medical valve 10 has an internal valving mechanism (not shown), the tip of medical instrument 40 may pass through a portion of the inlet seal 210 and aperture 220 and actuate/open the internal valving mechanism.

As described above, the inlet seal 210 may be made from a resilient material that allows the inlet seal 210 to automatically return back to the normal (e.g., at rest) shape in the absence of pressure/force. In other words, as the medical practitioner 20 removes the medical instrument 40, the inlet seal 210 will begin to return to the at rest position shown in FIG. 3. Additionally, as the instrument 40 is withdrawn, the aperture 220 will also close, fluidly disconnecting the medical instrument 40 with the internal area of the valve 10.

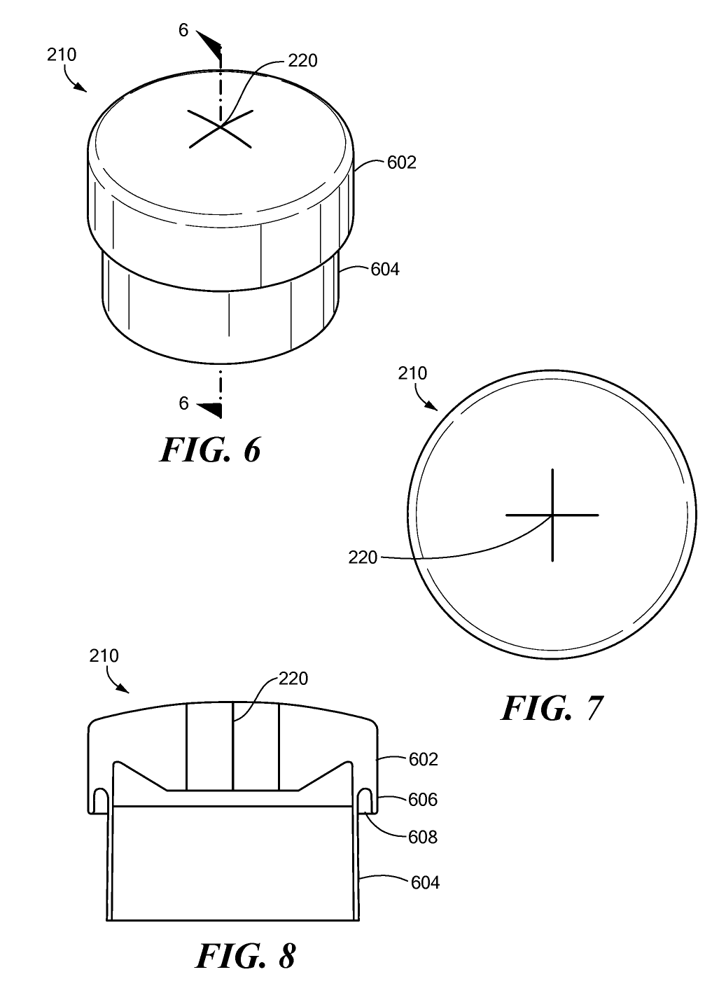

As best shown in FIGS. 6-8, the inlet seal 210 may have a body portion 602 and a sleeve portion 604. The body portion 602 may contain the aperture 220 and may be the portion that is raised above the exterior inlet face 260 of the inlet housing 240. The sleeve portion 604 may extend distally from the body portion 602 and conform to the inner diameter of the inlet housing 240. Additionally, the inlet seal 210 may also have a lip portion 606 that conforms to the outer diameter of the inlet housing 240. In such embodiments, the lip portion 606 and the sleeve portion 604 can create an annular recess 608 that correspond to the inlet housing 240 and the exterior inlet face 260. The annular recess 608 increases the surface area of the inlet seal 210, which improves chemical bonding with the inlet housing 240.

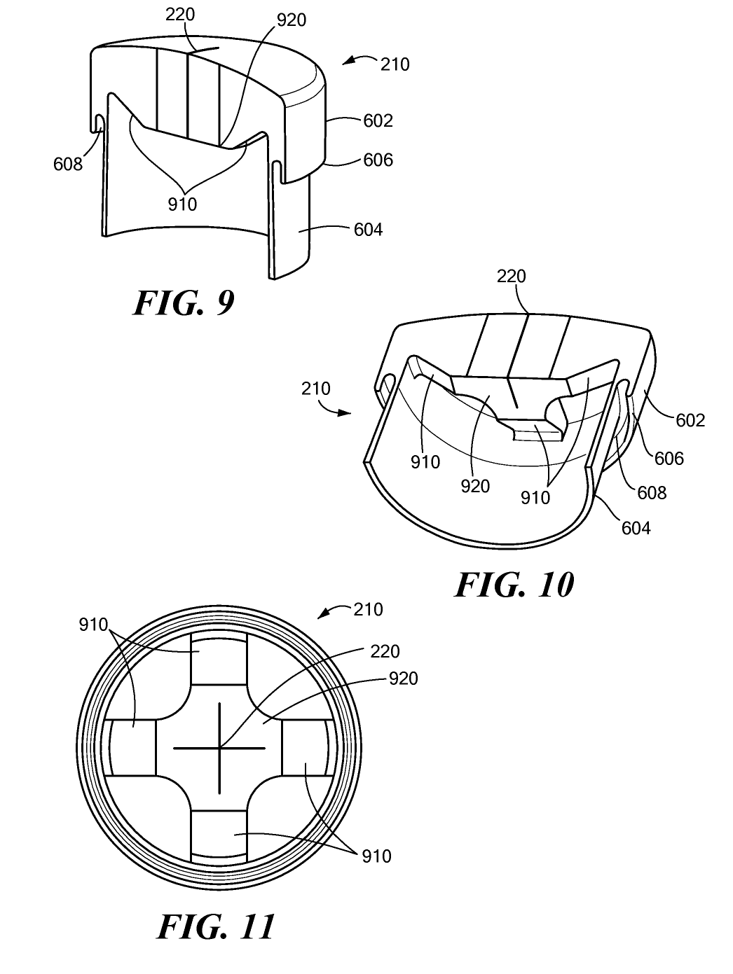

As mentioned above and as shown in FIGS. 9-11, the inlet seal 210 can have internal geometry that helps the aperture 220 remained sealed and helps prevent microbial ingress. In particular, the internal geometry may include cross-bars 910. The cross-bars 910 may extend across the inner diameter of the inlet seal 210 and create a plateau 920 surrounding the distal side of the aperture 220. In some embodiments, the cross-bars 910 may be angled such that the cross-bars 910 are thicker at the plateau 920 (e.g., the thickness of the cross-bars 910 increases as the cross-bars 910 approach the aperture 220). The thicker area surrounding the aperture 220 (e.g., the plateau 920) increases the sealing surface of the aperture 220 (e.g., through the thickness of the plateau 920), which, in turn, improves the aperture's ability to remained sealed and prevent microbial ingress.

The medical valve 10, the inlet housing 240, and the inlet seal 210 may be manufactured in a variety of ways. However, in preferred embodiments of the present invention, the inlet housing 240 and the inlet seal 210 are manufactured such that they form a single piece. In particular, the inlet seal and the inlet housing can be manufactured in a “two-shot”, “over-mold”, or “co-injection” process, collectively known as multimaterial molding. As known by those in the art, the multimaterial molding process creates one piece formed with two materials (i.e., the elastomeric inlet seal material and the material forming the rigid inlet housing) that are chemically bonded to one another.

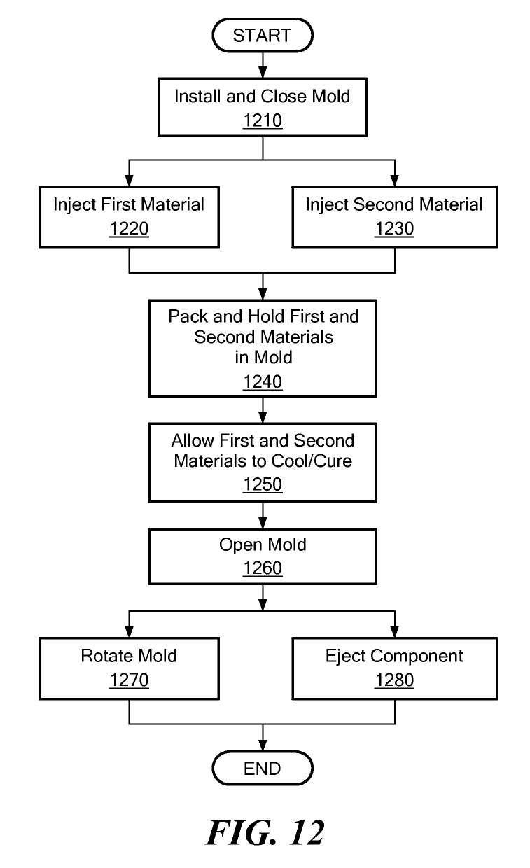

It is important to note that multimaterial parts may be made using a single station or a multi-station molding process. FIG. 12 shows steps to an exemplary process for multimaterial molding. It will be apparent to those skilled in the art that many variations to the disclosed process are possible for achieving a multimaterial part. Prior to beginning the molding of a multimaterial part, it is assumed that a multimaterial mold has been properly designed to result in the intended end product (e.g. the inlet with raised seal 310, FIG. 3). The mold is also understood to be properly installed in an appropriately sized press to complete the molding operation. Furthermore, the press is understood to be loaded with proper materials for creating the desired product, and the materials have been accurately conditioned prior to molding.

Upon successful set-up of the mold and molding equipment described above, the user and/or the molding equipment closes the mold (step 1210) to begin the multimaterial molding process. After the mold is closed, materials (e.g., a first material and a second material) are injected into the mold to begin part formation (steps 1220 and 1230). In the case of “two-shot” molding and “over-molding,” the mold may have two portions and each material may be injected into separate portions of the mold. Additionally, material injection may be performed in independent injection steps (e.g., one step for the first material and a second step for the second material), and each portion of the multimaterial product may be formed individually. For example, the inlet housing 240 may be formed within the first portion of the mold during the first injection step, and the inlet seal 210 may be formed in the second portion of the mold during a second injection step. In the case of “co-injection molding” both materials may be injected into the same portion of the mold simultaneously to form the multimaterial product (e.g., a product with both materials).

Following the injection processes, the materials may then be packed and held in the mold cavity (step 1240), and allowed to cool or cure (step 1250). For example, the plastic component (e.g., inlet 240) may undergo a cooling phase in the mold while the resilient seal 210 may undergo a curing cycle. The user and/or the molding equipment may then open the mold (step 1260), and the completed inlet with raised seal 310 may be ejected from the mold (step 1280). As discussed above, in two-shot and over-molding processes, the inlet 240 may be formed in the first portion of the mold. In some embodiments, the inlet 240 may remain in the mold and may be rotated (or otherwise transferred) (step 1270) into the second portion of the mold so that the second material (e.g., for the inlet seal 210) may be injected and the inlet seal 210 formed. The entire process may then repeat to create additional components. As explained in further detail below, use of a multimaterial molding process can significantly minimize the possibility of fluid leaking between the inlet seal 210 and inlet housing 240.

It is important to note that the multimaterial molding process and the resulting chemical bond described above is unlike many over-molding processes that create only a mechanical bond between the components. For example, interference over-molding creates a mechanical bond between the components. The mechanical bond/interference may be created in a variety of ways including, but not limited to sizing the components such that the inner component is larger than the outer component or otherwise creating friction or mechanical interference between the components. The bond/interference between components manufactured using an interference over-mold process is purely mechanical. Interference over-molding does not provide any chemical bond between the components, which can significantly minimize the possibility of fluid leaking between the inlet seal 210 and inlet housing 240.

Although providing benefits, the multimaterial molding process provides challenges during the manufacture of medical valves. For example, the rate and quantity of shrinkage of the materials (e.g., the resilient inlet seal material and the rigid inlet housing material) may differ causing unwanted and/or problematic forces on one or more of the components. These forces can cause the aperture within the inlet seal to open, despite the valve 10 being in the “closed” position. For example, if the rigid inlet housing material shrinks less than the resilient inlet material, forces may build up in the inlet seal 210, causing the aperture 220 in the inlet seal 210 to open, even when the valve 10 is in the closed mode. More specifically, in one exemplary embodiment the resilient seal may be molded in silicone with a shrink rate of 2.3% on an inlet molded in polycarbonate with a shrink rate of 0.006 to 0.008″ per inch or 0.7%. Therefore, upon cutting the resilient seal to form an aperture, an opening may form at the center of the aperture, on the order of 0.007″. It should be noted in this example, that the outer diameter of the inlet is 0.265″ and the aperture is cut at a length of 0.125″. Various embodiments of the present invention overcome many of the challenges associated with multimaterial molding processes.

Specifically, as discussed above, at least a portion (e.g., the body portion 602) of the inlet seal 210 may be located above the inlet housing 240 and the exterior inlet face 260. Such an orientation allows the resilient inlet seal material to cool, cure, and shrink independently from the rigid inlet housing material (e.g., the inlet seal 210 is not constrained by the inlet housing 240). Accordingly, embodiments of the present invention avoid the unwanted and problematic forces described above.

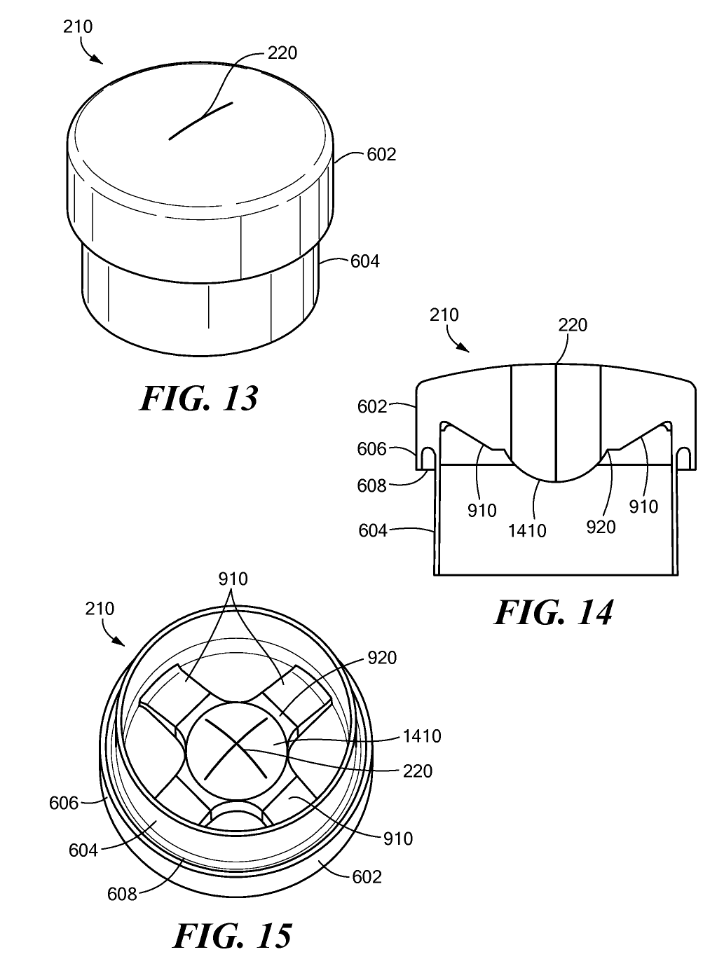

Although the aperture 220 is described and shown above as being multiple slits in the form of a cross, other aperture 220 orientations are within the scope of the present inventions. For example, as shown in FIG. 13, the aperture 220 may be a single slit. Alternatively, the aperture 220 can have more than two slits in the shape of a star or asterisk. Instead of one or more slits, as mentioned above, the aperture 220 may also be a pinhole. It is important to note that the aperture 220 (e.g., the one or more slits or pinhole) may be formed, for example, using traditional cutting means (e.g., razor blade, knife, etc.), piercing with a needle, or ultrasonic cutting methods. Additionally or alternatively, the aperture 220 could also be formed in-mold during or after the injection molding process.

As shown in FIGS. 14 and 15, some embodiments of the present invention may also include a dome 1410 located on the plateau 920 of the internal geometry. The dome 1410 may be located at the center of the plateau 920 portion so that the aperture 220 is located within the dome 1410 (see FIG. 15). The dome 1410 further increases the thickness of the inlet seal 210 at the slit plane. In other words, the dome 1410 further increases the aperture's 220 resistance to microbial ingress.

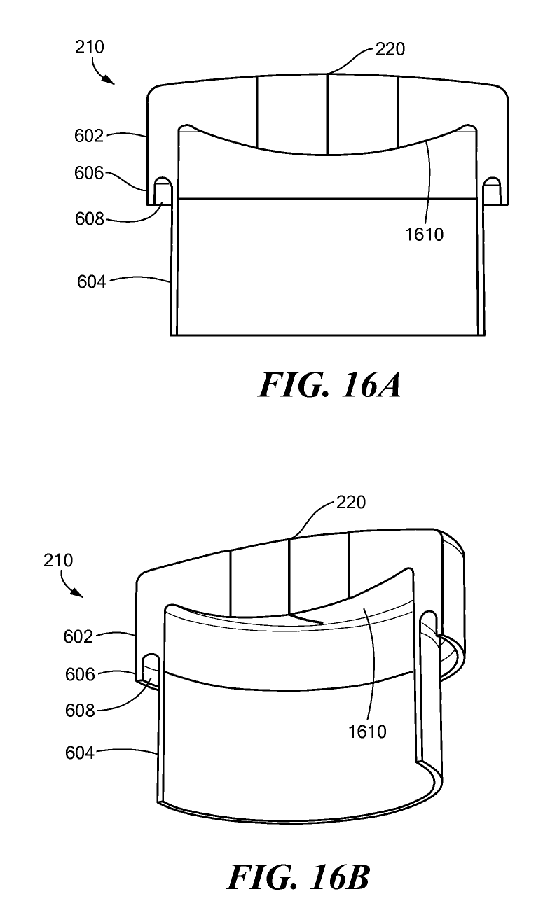

It is important to note that the internal geometry of the inlet seal 210 is not limited to the cross-bar and plateau orientation described above. For example, instead of the angled cross-bars 910 shown in FIGS. 9 and 10, the internal geometry may be an area of varying thickness (e.g., a dome 1610) that increases towards the aperture 220, such as shown in FIGS. 16A and 16B. Such an orientation increases the thickness of the inlet seal 210 at the aperture 220 and provides the benefits described above with respect to FIGS. 9 and 10. Additionally, the gradual increase in thickness from the edge of the inlet seal 210 to the center allows for greater stretch at the outer diameter (e.g., edge of the inlet seal 210), while still supporting the center of the inlet seal 210.

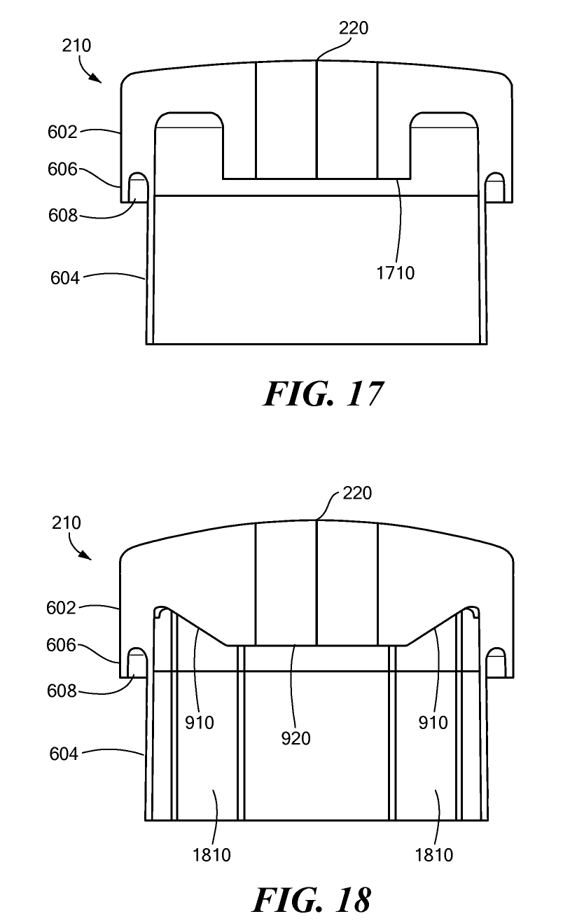

Although the embodiments described above have internal geometry including cross-bars or areas of varying thicknesses (e.g., domes), other embodiments of the present invention do not include such internal geometry. For example, as shown in FIG. 17, the internal geometry of the inlet seal 210 can simply be a single central protrusion 1710. The single central protrusion 1710 increases the thickness of the inlet seal 210 around the aperture 220 to help keep the aperture 220 closed when the valve 10 is at rest (e.g., in the closed mode). Additionally, because the remaining area is thin (e.g., as compared to the central protrusion 1710), the inlet seal 210 may easily stretch and deform upon insertion of the medical instrument 40.

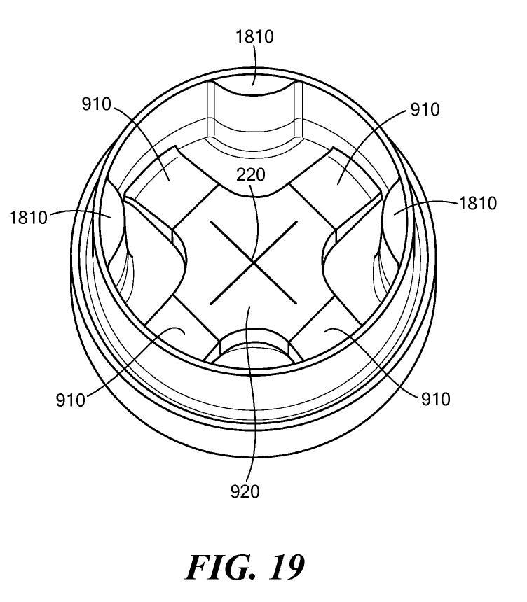

Additionally or alternatively, as shown in FIGS. 18-19, any of the above described embodiments of the inlet seal 210 may also include ribs 1810 located along the inner sidewalls of the inlet seal 210. As the medical practitioner 20 inserts the medical instrument 40, the ribs 1810 support the medical instrument 40 and the portions of the inlet seal 210 that deform into the inlet housing 240. By supporting these components, the ribs 1810 are able to reduce the stress on the ends of the aperture 210 (e.g., the ends of the slits). The ribs 1810 also help to support and strengthen the body portion 602 when at rest, further increasing the inlet seal's resistance to microbial ingress.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A seal for a medical valve having a housing and an inlet, the seal comprising:
a seal portion over the inlet of the valve, the seal portion having a normally closed aperture therethrough, the aperture located proximal to the inlet; and
a sleeve portion extending distally from the seal portion, at least a part of the sleeve portion being chemically bonded to the inlet.

2. A seal according to claim 1, wherein at least a part of the sleeve portion is chemically bonded using a multimaterial molding process.

3. A seal according to claim 2, wherein the multimaterial molding process is selected from a group consisting of two-shot, over-mold, and co-injection manufacturing processes.

4. A seal according to claim 1, further comprising at least one cross member extending across a diameter of the seal portion on a distal surface of the seal portion, the cross member configured to increase resistance to microbial ingress.

5. A seal according to claim 1, further comprising a lip portion co-centric with the sleeve portion and configured to extend distally along an outer diameter of the inlet.

6. A seal according to claim 1, wherein the seal portion deforms into the inlet after insertion of a medical instrument.

7. A seal according to claim 1, wherein the seal portion is swabbable.

8. A seal according to claim 1, wherein the aperture is a slit.

9. A seal according to claim 1, wherein the aperture is a pinhole.

10. A seal according to claim 1 wherein the sleeve portion is configured to be disposed within an inner diameter of the inlet.

11. A seal according to claim 1 wherein at least a part of the seal portion is chemically bonded to the inlet.

12. A seal according to claim 1, wherein the seal portion is resilient.

13. A seal according to claim 1, wherein the sleeve portion is resilient.

14. A seal for a medical valve having a housing and an inlet, the seal comprising:
a seal portion chemically bonded to the housing, the seal portion disposed over and raised above the inlet of the valve; and
a normally closed aperture extending through the seal portion, the aperture located proximal to the inlet.

15. A seal according to claim 14, further comprising:
a sleeve portion extending distal to the seal portion and configured to be disposed within an inner diameter of the inlet, at least a portion of the sleeve portion being chemically bonded to the inlet.

16. A seal according to claim 15, wherein the seal portion and the sleeve portion are chemically bonded using a multimaterial molding process.

17. A seal according to claim 16, wherein the multimaterial molding process is selected from a group consisting of two-shot, over-mold, and co-injection manufacturing processes.

18. A medical valve according to claim 14, wherein the seal is resilient.

19. A medical valve having an open mode that permits fluid flow, and a closed mode that prevents fluid flow, the medical valve comprising:
an inlet housing having an inlet;
a resilient seal member chemically bonded to the inlet housing and having a normally closed aperture, at least a portion of the seal member extending proximal to the inlet such that the normally closed aperture is proximal to the inlet.

20. A medical valve according to claim 19, wherein the inlet housing and the seal member are chemically bonded using a multimaterial molding process.

21. A medical valve according to claim 20, wherein the multimaterial molding process is selected from a group consisting of two-shot, over-mold, and co-injection manufacturing processes.

22. A medical valve according to claim 19, wherein the seal member includes:
a seal portion disposed over the inlet of the valve; and
a sleeve portion extending distally from the seal portion, the sleeve portion having an outer diameter that is chemically bonded to an inner diameter of the inlet.

23. A method for producing a medical valve comprising:
injecting a first material into a mold to form an inlet housing of the medical valve;
injecting a second material into the mold to form a seal member of the medical valve;
allowing the first and second materials to cure and cool within the mold;
removing the inlet housing and seal member from the mold, at least a portion of the seal member being chemically bonded to the inlet housing; and
forming an aperture through the seal member, the aperture being normally closed and located proximal to the inlet.

24. A method according to claim 23, wherein the mold has a first portion and a second portion, the first material being injected into the first portion and the second material being injected into the second portion of the mold.

25. A method according to claim 24 wherein the first and second materials are allowed to cure at different times.

26. A method according to claim 23, wherein the first and second materials are injected substantially simultaneously into the same portion of the mold.
